(19) United States
(12) Patent Application Publication
(10) Pub. No.: US 2022/0296133 A1
(43) Pub. Date: Sep. 22, 2022

(54) Methods Circuits Devices Systems and Machine Executable Code for Glucose Event Detection

(71) Applicant: CaloSense Ltd., Netanya (IL)

(72) Inventor: Nitzan Shenar, Netanya (IL)

(73) Assignee: CaloSense Ltd., Netanya (IL)

(21) Appl. No.: 17/700,728

(22) Filed: Mar. 22, 2022

Related U.S. Application Data
(60) Provisional application No. 63/163,965, filed on Mar. 22, 2021.

(51) Int. Cl. A61B 5/145; G06N 3/04; G06N 3/08; A61B 5/024

(52) U.S. Cl. CPC A61B 5/14532; G06N 3/049; G06N 3/08; A61B 5/024

(57) ABSTRACT

Disclosed are methods, circuits, devices, systems and functionally associated machine executable code for glucose event detection. A system for glucose event detection includes a recursive neural network (RNN) model for generating, for a monitored subject, blood glucose level (BGL) output streams for respective, system fed, heart beats per minute (BPM) input streams of a monitored subject. A supervised training mechanism, for training the artificial recurrent neural network (RNN) model, compares model generated blood glucose level (BGL) output streams to time-aligned blood glucose level (BGL) output streams from a continuous glucose monitoring (CGM) device concurrently monitoring the same subject.

### FIELD OF THE INVENTION

The present invention generally relates to the field of glucose level monitoring. More specifically, the present invention relates to methods, circuits, devices, systems and machine executable code for glucose event detection.

### BACKGROUND

Diabetes is one of the biggest global health crises of the 21st century, with an estimation of 1B diabetic & pre-diabetics globally. Only a change of lifestyle can reverse condition effectively. Pre-Ds are scared and confused but mostly feel helpless—and in 5-6 years 30% of them will develop diabetes. Estimating the total costs of diagnosed diabetics has risen to $327 billion in 2008, in the USA alone.

There remains a need, in the field of glucose level monitoring, for solutions that may detect glucose level events and trends of a monitored subject based on data gathered by noninvasive sensors and wearables including same.

### SUMMARY OF THE INVENTION

Embodiments of the present invention include methods, circuits, devices, systems and machine executable code for glucose event detection.

There may be provided, in accordance with some embodiments, a glucose event detection system, comprising an artificial recurrent neural network (RNN) architecture of long short-term memory (LSTM) cells, configured to generate, for a monitored subject, variable blood glucose level (BGL) output streams predictions for respective, system fed, variable heart beats per minute (BPM) input streams of the monitored subject.

A system in accordance with embodiments, may further comprise a computer readable medium including instructions for a supervised training mechanism for, iteratively: (1) feeding to the artificial RNN model, as input data, variable BPM input streams of the monitored subject, for the model to generate corresponding variable BGL model output streams predictions; (2) applying a loss function for measuring the “error” in the model's BGL output streams predictions, relative to time-aligned and labeled variable BGL output streams from a continuous blood glucose monitoring (CGM) device concurrently monitoring the subject; and/or (3) updating weight values of the RNN model based on the loss function measured “error”, to gradually reduce that error.

The glucose event detection system, in accordance with embodiments, may include a photoplethysmogram (PPG) based device for monitoring the heart rate of the subject and generating the timestamped variable BPM input streams utilized for: (1) training of the artificial RNN model; and/or (2) feeding to the trained artificial RNN model to generate respective variable blood glucose level (BGL) predictions output streams, based thereof.

The RNN model's blood glucose level (BGL) predictions output streams may be analyzed to detect specific value/values-combination/trends indicative of a glucose event. Detection of a glucose event may trigger the relaying of a notification, wherein the notification, in accordance with some embodiments, may include one or more RNN model's blood glucose level (BGL) predictions associated with the detected event.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings:

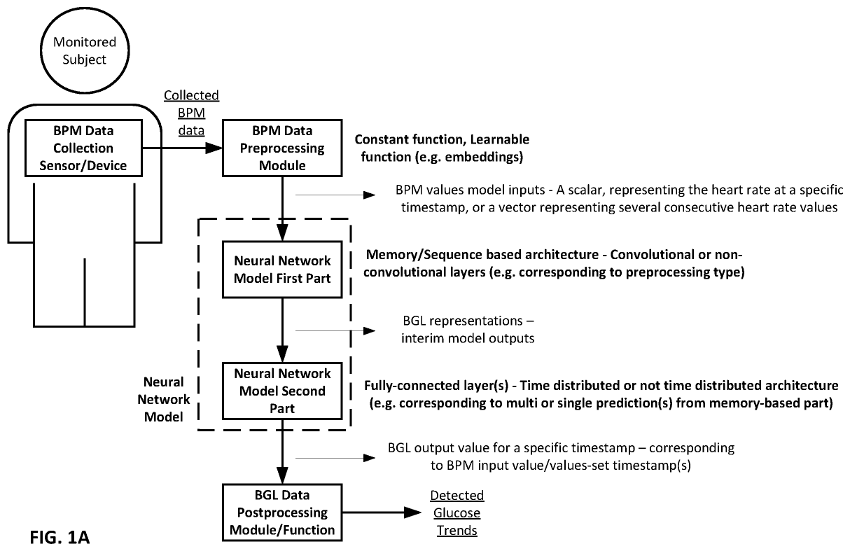

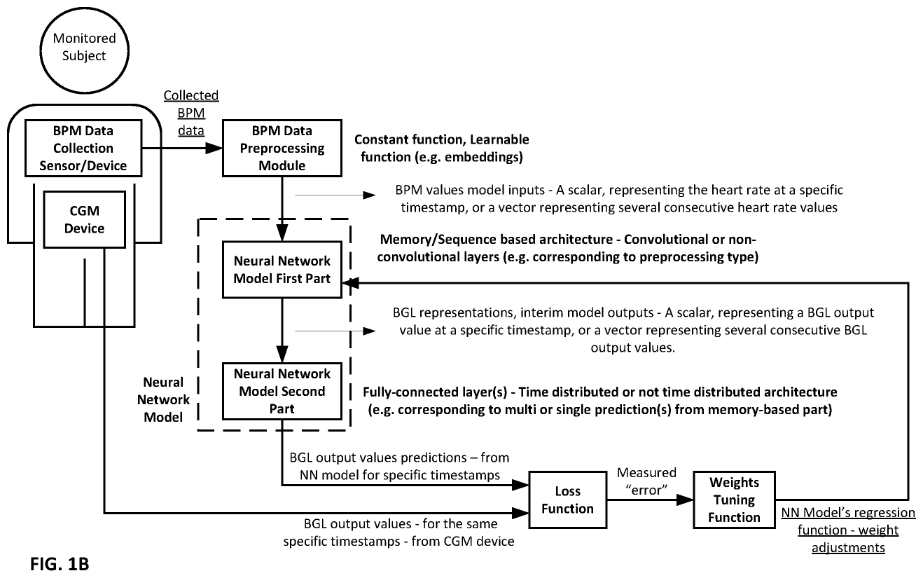

In FIG. 1A, there is shown a block diagram of an exemplary system for glucose event detection including a recursive neural network (RNN) model, in accordance with some embodiments of the present invention;

In FIG. 1B, there is shown a block diagram of an exemplary system for glucose event detection including a supervised training mechanism, in accordance with some embodiments of the present invention;

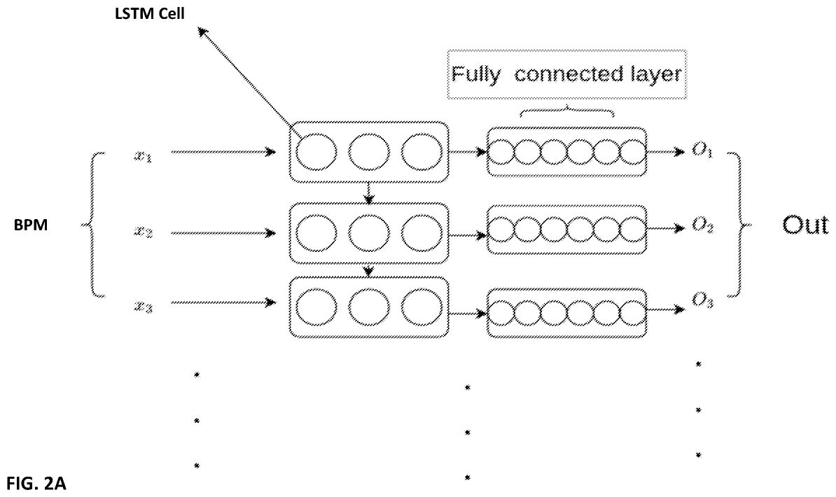

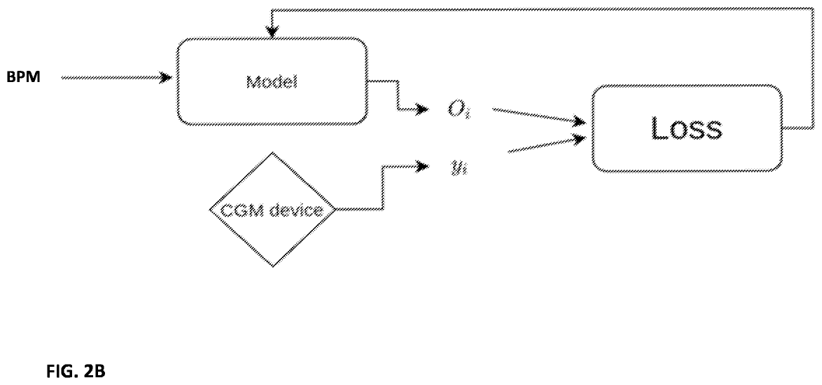

In FIG. 2A, there is shown a schematic block diagram exemplifying in further detail a recursive neural network (RNN) model for generating blood glucose level (BGL) output streams, in accordance with some embodiments of the present invention;

In FIG. 2B, there is shown a schematic block diagram exemplifying in further detail a a supervised training mechanism, in accordance with some embodiments of the present invention;

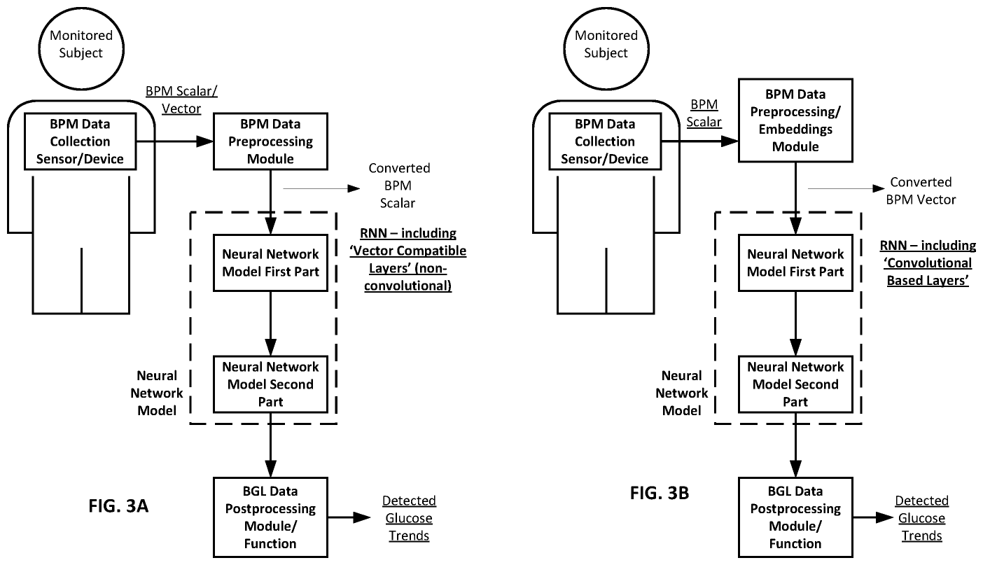

In FIG. 3A there is shown, an exemplary glucose event detection system in accordance with embodiments, wherein a first pre-processing scheme is implemented;

In FIG. 3B there is shown, an exemplary glucose event detection system in accordance with embodiments, wherein a second pre-processing scheme is implemented;

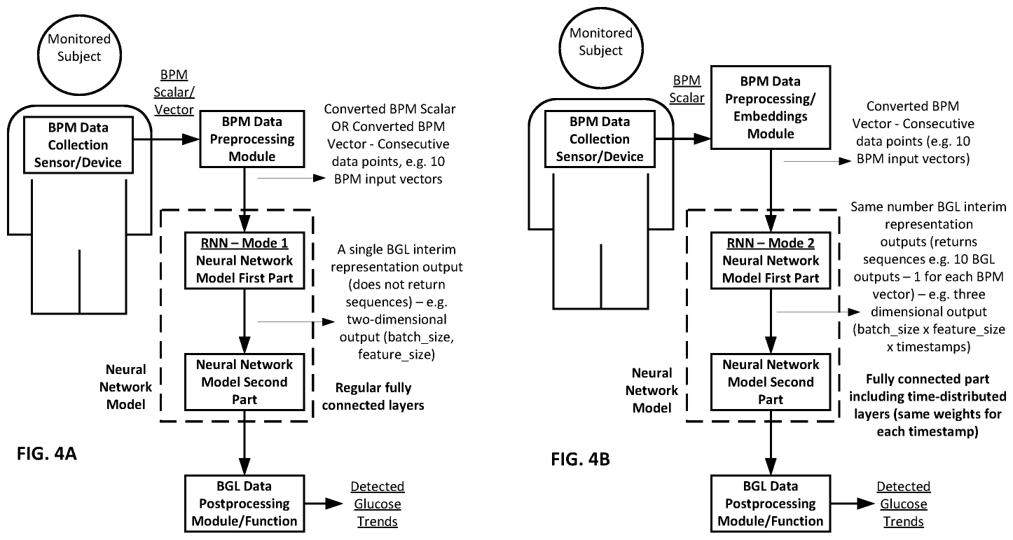

In FIG. 4A there is shown, an exemplary glucose event detection system in accordance with embodiments, wherein a first RNN mode is implemented; and

In FIG. 4B there is shown, an exemplary glucose event detection system in accordance with embodiments, wherein a second RNN mode is implemented.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals or element labeling may be repeated among the figures to indicate corresponding or analogous elements.

### DETAILED DESCRIPTION

In the following detailed description, numerous specific details are set forth in order to provide a thorough understanding of some embodiments. However, it will be understood by persons of ordinary skill in the art that some embodiments may be practiced without these specific details. In other instances, well-known methods, procedures, components, units and/or circuits have not been described in detail so as not to obscure the discussion.

Unless specifically stated otherwise, as apparent from the following discussions, it is appreciated that throughout the specification discussions utilizing terms such as “processing”, “computing”, “calculating”, “determining”, or the like, may refer to the action and/or processes of a computer, computing system, computerized mobile device, or similar electronic computing device, that manipulate and/or transform data represented as physical, such as electronic, quantities within the computing system's registers and/or memories into other data similarly represented as physical quantities within the computing system's memories, registers or other such information storage, transmission or display devices.

In addition, throughout the specification discussions utilizing terms such as “storing”, “hosting”, “caching”, “saving”, or the like, may refer to the action and/or processes of ‘writing’ and ‘keeping’ digital information on a computer or computing system, or similar electronic computing device, and may be interchangeably used. The term “plurality” may be used throughout the specification to describe two or more components, devices, elements, parameters and the like.

Some embodiments of the invention, may for example take the form of an entirely hardware embodiment, an entirely software embodiment, or an embodiment including both hardware and software elements. Some embodiments may be implemented in software, which includes but is not limited to, any combination of: firmware, resident software, microcode, or the like. Some embodiments may be implemented in hardware, which includes but is not limited to, any combination of: a processor, memory and data storage components, a power source, communication circuitry, I/O interfaces, cards and devices, programmable arrays, systems on chip, or the like. Some embodiments may be implemented using a combination of hardware and software, which includes but is not limited to, any combination of the above hardware and software types and components.

Furthermore, some embodiments of the invention may take the form of a computer program product accessible from a computer-usable or computer-readable medium providing program code for use by or in connection with a computer or any instruction execution system. For example, a computer-usable or computer-readable medium may be or may include any apparatus that can contain, store, communicate, propagate, or transport the program for use by or in connection with the instruction execution system, apparatus, or device, for example a computerized device running a web-browser.

In some embodiments, the medium may be an electronic, magnetic, optical, electromagnetic, infrared, or semiconductor system (or apparatus or device) or a propagation medium. Some demonstrative examples of a computer-readable medium may include a semiconductor or solid-state memory, magnetic tape, a removable computer diskette, a random access memory (RAM), a read-only memory (ROM), a rigid magnetic disk, and an optical disk. Some demonstrative examples of optical disks include compact disk-read only memory (CD-ROM), compact disk-read/write (CD-R/W), and DVD.

In some embodiments, a data processing system suitable for storing and/or executing program code may include at least one processor coupled directly or indirectly to memory elements, for example, through a system bus. The memory elements may include, for example, local memory employed during actual execution of the program code, bulk storage, and cache memories which may provide temporary storage of at least some program code in order to reduce the number of times code must be retrieved from bulk storage during execution. The memory elements may, for example, at least partially include memory/registration elements on the user device itself.

In some embodiments, input/output or I/O devices (including but not limited to keyboards, displays, pointing devices, etc.) may be coupled to the system either directly or through intervening I/O controllers. In some embodiments, network adapters may be coupled to the system to enable the data processing system to become coupled to other data processing systems or remote printers or storage devices, for example, through intervening private or public networks. In some embodiments, modems, cable modems and Ethernet cards are demonstrative examples of types of network adapters. Other suitable components may be used.

Functions, operations, components and/or features described herein with reference to one or more embodiments, may be combined with, or may be utilized in combination with, one or more other functions, operations, components and/or features described herein with reference to one or more other embodiments.

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “includes”, “including”, “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

The present disclosure is to be considered as an exemplification of the invention and, is not intended to limit the invention to the specific embodiments illustrated by the figures or description below.

Embodiments of the present invention include a glucose event detection system, comprising an artificial recurrent neural network (RNN) architecture of long short-term memory (LSTM) cells, configured to generate, for a monitored subject, variable blood glucose level (BGL) output streams predictions for respective, system fed, variable heart beats per minute (BPM) input streams of the monitored subject.

A system in accordance with embodiments, may further comprise a computer readable medium including instructions for a supervised training mechanism for, iteratively: (1) feeding to the artificial RNN model, as input data, variable BPM input streams of the monitored subject, for the model to generate corresponding variable BGL model output streams predictions; (2) applying a loss function for measuring the “error” in the model's BGL output streams predictions, relative to time-aligned and labeled variable BGL output streams from a continuous blood glucose monitoring (CGM) device concurrently monitoring the subject; and/or (3) updating weight values of the RNN model based on the loss function measured “error”, to gradually reduce that error.

The glucose event detection system, in accordance with embodiments, may include a photoplethysmogram (PPG) based device for monitoring the heart rate of the subject and generating the time stamped variable BPM input streams for: (1) training of the artificial RNN model; and/or (2) feeding to the trained artificial RNN model to generate respective variable blood glucose level (BGL) output streams predictions based thereof.

The RNN model's blood glucose level (BGL) predictions output streams may be analyzed/postprocessed to detect specific value/values-combination/trends indicative of a glucose event. Detection of a glucose event may trigger the relaying of a notification, wherein the notification, in accordance with some embodiments, may include one or more RNN model's blood glucose level (BGL) predictions associated with the detected event.

According to some embodiments, the glucose detection system, may further include, or receive output values from, an accelerometer concurrently monitoring the subject. Accelerometer output values may, in accordance with some embodiments, be utilized for mitigating false-positive alerts that are based on the neural network (NN) model's BGL level outputs.

Accelerometer output values may be analyzed to detect ‘higher than normal’ values occurring concurrently with glucose events detected based on the NN model's BGL values outputs. At least part of the NN model's detected glucose events—time overlapping with ‘higher than normal’ accelerometer values (indicative of higher physical activity of the monitored subject and higher BPM values caused thereby)—may not be labeled/regarded as glucose events, due to their overlapping time occurrence with higher physical activity accelerometer-based indications.

The CGM device, in accordance with embodiments, may take the form a noninvasive CGM device, such as, but not limited to, a wearable CGM device (e.g., a ‘freestyle libre’ device/system).

According to some embodiments, the monitored subject's BPM inputs to the RNN may take, or be preprocessed to take, the structure/form of a scalar—representing the heart rate value of the subject at a specific timepoint; and/or, of a vector—representing several consecutive heart rate values of the subject.

A first network section/part of an RNN in accordance with embodiments, may consist of a memory-based architecture, for connecting between BPM input series and BGL output series. The first network section/part may take the form of a sequence-based architecture, for example, an architecture of one or more layers of LSTM cells/neurons.

A second network section/part of an RNN in accordance with embodiments, may consist of one or more fully-connected layers, for yielding a final output of the model—a predicted BGL value of the monitored subject at a specific timepoint—based on the output of the memory-based section/part.

According to some embodiments, the input (heart rate data) may be pre-processed prior to being fed to the network. Input data pre-processing schemes, in accordance with embodiments, may for example include: the application of a constant function, such as calculation of the logarithm of the raw input values; and/or, a more complicated learnable function, such as embeddings.

According to the specific pre-processing scheme utilized, the memory-based section/part of the network may include either convolutional or non-convolutional layers.

The memory-based section/part, in accordance with embodiments, may return/output a single prediction over multiple input data timestamps; or return/output multiple predictions, corresponding to multiple input data timestamps. The return/output of either single or multiple predictions, may determine whether a time distributed (for single prediction), or not a time distributed (for multiple predictions), architecture is utilized for the second section/part of the network.

According to some embodiments, the output of the network may be post-processed, before obtaining the final BGL output values, to improve the quality/accuracy of the results.

According to some embodiments, the NN model's BGL values outputs may be analyzed for the detection of glucose events, based on a classification scheme, including: (1) detection of an anomaly, based on a peak(s) in the NN model's BGL values outputs—wherein a peak may be defined as an increase beyond a threshold size of the BGL values outputs, the increase occurring within a time period shorter than a threshold length time period; (2) classifying the anomaly as either a food intake related or physical activity related anomaly; and/or (3) reacting (e.g. triggering an alert/notification, labeling as a glucose event) to anomalies classified as food intake related.

In FIG. 1A, there is shown a block diagram of an exemplary system for glucose event detection including a recursive neural network (RNN) model, in accordance with some embodiments of the present invention, for generating, for a monitored subject, blood glucose level (BGL) output streams for respective, system fed, heart beats per minute (BPM) input streams of the monitored subject.

In the exemplary figure, BPM data from a BPM sensor/or device, monitoring a subject, is fed into a preprocessing module to generate BPM values inputs to the shown neural network model. The first part of the trained model connects the BPM values inputs to corresponding BGL interim representation outputs. The second part of the trained model generates time specific BGL output values for corresponding BPM timestamps, based on outputs from the first part. Outputs of the second part of the trained model are postprocessed to detect glucose trends, for example, based on changes along time (e.g. a sudden peak(s)) in the model's output values.

In FIG. 1B, there is shown a block diagram of an exemplary system for glucose event detection including a supervised training mechanism, in accordance with some embodiments of the present invention, for training the artificial recurrent neural network (RNN) model of FIG. 1A, based on the comparison of model generated blood glucose level (BGL) output streams to time-aligned blood glucose level (BGL) output streams from a continuous glucose monitoring (CGM) device.

The final neural network model's BGL output values are fed, along with time respective (i.e. same specific timestamps) BGL output values received from a CGM device monitoring the subject concurrently with the BPM sensor/device—into a loss function component of the supervised training mechanism for comparison. Deltas between the model's BGL output values and time respective CGM device output values are calculated to determine a measured error of the model's predictions. The measured errors are relayed to a weights tuning function of the supervised training mechanism to determine, based on a regression function using the calculated deltas as input parameters, weight adjustments values for the NN's layers and/or cells/neurons.

In FIG. 2A, there is shown a schematic block diagram exemplifying in further detail a recursive neural network (RNN) model for generating blood glucose level (BGL) output streams, including the RNN's components and component interrelations, in accordance with some embodiments of the present invention.

In the figure, a series/vector of BPM input values along time—X₁, X₂, X₃ . . . —are fed into the RNN model. The RNN model is shown to include a first part, consisting of a memory-based architecture including a layer(s) of LSTM cells/neurons for generating predicted BGL output value(s) corresponding to the model fed BPM inputs. The RNN model is shown to further include a second part consisting of a fully connected layer(s) for yielding final BGL value predictions—O₁, O₂, O₃ . . . —for the series/vector of BPM input values, based on the outputs of the first, memory-based architecture, part of the model.

In FIG. 2B, there is shown a schematic block diagram exemplifying in further detail a a supervised training mechanism for training the recursive neural network (RNN) model of FIG. 2A, in accordance with some embodiments of the present invention.

In the figure, a final BGL value prediction of the model Oᵢ—generated based on BPM model input values of the monitored subject—is shown be fed to a loss function, along with a time-aligned/time-synched BGL value measurement Y, from a CGM device concurrently monitoring the same subject. The error, between the BPM based model prediction and the CGM measurement, is calculated by the loss function and the RNN model's weights are tuned based thereof, to gradually minimize the error, thereby training the model.

According to some embodiments, a glucose event detection system may implement a first pre-processing scheme, of the RNN's inputs, that converts each BPM scalar to another scalar or a BPM vector to a scalar. The architecture of the first NN part (RNN part), may accordingly include “vector compatible layers” (non-convolutional).

In FIG. 3A there is shown, an exemplary glucose event detection system in accordance with embodiments, wherein a first pre-processing scheme is implemented.

According to some embodiments, a glucose event detection system may implement a second pre-processing scheme, of the RNN's inputs, that converts each BPM scalar to a vector (e.g. embeddings). The architecture of the first NN part (RNN part), may accordingly include convolutional based layers, due to the extra dimension that was created in the pre-processing part.

In FIG. 3B there is shown, an exemplary glucose event detection system in accordance with embodiments, wherein a second pre-processing scheme is implemented.

According to some embodiments, the RNN of the glucose event detection system may be structured, and operate, in a first RNN mode, wherein a number of consecutive data points may be converted to a single output (i.e. does not return sequences).

The first RNN mode, may return a single BGL output for multiple (e.g. 10) BPM timestamp inputs (vectors). The RNN may accordingly include regular fully connected layers, to generate and provide a two-dimensional output (batch_size, feature_size).

According to some embodiments, the RNN of the glucose event detection system may be structured, and operate, in a second RNN mode, wherein a number of consecutive data points may be converted to a similar number of outputs (i.e. returns sequences).

The second RNN mode, may return a similar number of BGL outputs for multiple (e.g. 10) BPM timestamp inputs—a single prediction for each BPM datapoint. The RNN may accordingly include time-distributed fully connected layers, to generate and provide a three-dimensional output (batch_size, feature_size, timestamps).

A datapoint, in accordance with some embodiments, may comprise of a vector including multiple (e.g. 15) BPM values, or alternatively, a single BPM value.

In FIG. 4A there is shown, an exemplary glucose event detection system in accordance with embodiments, wherein a first RNN mode is implemented.

In FIG. 4B there is shown, an exemplary glucose event detection system in accordance with embodiments, wherein a second RNN mode is implemented.

According to some embodiments of the present invention, a system for glucose event detection may comprise an artificial recurrent neural network (RNN) architecture of long short-term memory (LSTM) cells, configured to generate, for a monitored subject, variable blood glucose level (BGL) output streams predictions for respective, system fed, variable heart beats per minute (BPM) input streams of the monitored subject.

According to some embodiments, the system may further comprise a computer readable medium including instructions for a supervised training mechanism for, iteratively: (1) feeding to the artificial RNN architecture model, as input data, variable BPM input streams of the monitored subject, for the model to generate corresponding variable BGL model output streams predictions; (2) applying a loss function for measuring the “error” in the model's BGL output streams predictions, relative to time-aligned and labeled variable BGL output streams from a continuous blood glucose monitoring (CGM) device concurrently monitoring the subject; and (3) updating weight values of the RNN model based on the loss function measured “error”, to gradually reduce that error.

According to some embodiments, the system may further comprise a photoplethysmogram (PPG) based device for monitoring the heart rate of the subject and generating the time stamped variable BPM input streams for: (1) training of the artificial RNN model; and (2) feeding to the trained artificial RNN model, to generate respective variable blood glucose level (BGL) output streams predictions based thereof.

According to some embodiments, the RNN model's blood glucose level (BGL) predictions output streams may be postprocessed to detect a specific value or values-trends indicative of a glucose event. A detection of a glucose event may trigger the relaying of a notification including one or more of the RNN model's blood glucose level (BGL) predictions associated with the detected event.

According to some embodiments, the system may further comprise an accelerometer concurrently monitoring the subject, wherein the accelerometer output values may be utilized for mitigating false-positive alerts detected within the RNN model's BGL level outputs.

According to some embodiments, the accelerometer output values may be analyzed to detect ‘higher than normal’ values occurring concurrently with glucose events detected within the RNN model's BGL values outputs. At least part of the RNN model's detected glucose events—time overlapping with ‘higher than normal’ accelerometer values—may be not-regarded/ignored/not-notified as glucose events, due to their overlapping time occurrence with higher physical activity indications that are based on the accelerometer output values.

According to some embodiments, the system's CGM device may be a noninvasive wearable CGM device.

According to some embodiments, the monitored subject's BPM inputs to the RNN model may be preprocessed to take the form of a scalar—representing the heart rate value of the subject at a specific timepoint.

According to some embodiments, the monitored subject's BPM inputs to the RNN model may be preprocessed to take the form of a vector—representing several consecutive heart rate values of the subject.

According to some embodiments, a first network section of the RNN may consist of a memory-based architecture, for connecting between BPM input series and BGL output series.

According to some embodiments, a second network section of the RNN may consist of one or more fully-connected layers, for yielding a final output of the model—a predicted BGL value of the monitored subject at a specific timepoint—based on the output of the memory-based section.

According to some embodiments, the variable heart beats per minute (BPM) input streams of the monitored subject may be pre-processed prior to being fed to the RNN by application of a logarithmic function on the raw input values.

According to some embodiments, the first network section of the RNN may include convolutional layers.

According to some embodiments, the variable heart beats per minute (BPM) input streams of the monitored subject may be pre-processed prior to being fed to the RNN by application of a learnable embeddings function.

According to some embodiments, the first network section of the RNN may include non-convolutional layers.

According to some embodiments, the memory-based section of the RNN, may output a single prediction over multiple input data timestamps.

According to some embodiments, the memory-based section of the RNN, may output multiple predictions, corresponding to multiple input data timestamps.

According to some embodiments, the RNN model's BGL values outputs may be analyzed for the detection of glucose events, based on a classification scheme, selected from the group including: (1) detection of an anomaly, based on one or more peaks in the RNN model's BGL values outputs—wherein a peak is defined as an increase beyond a threshold size of the BGL values outputs, the increase occurring within a time period shorter than a threshold length time period; (2) classifying the anomaly as either a food intake related or physical activity related anomaly; and (3) reacting only to anomalies classified as food intake related.

According to some embodiments, a glucose event detection method, may comprise feeding variable heart beats per minute (BPM) input streams of a monitored subject to an artificial recurrent neural network (RNN) architecture of long short-term memory (LSTM) cells, configured to generate, time-respective variable blood glucose level (BGL) output streams predictions for the fed variable heart beats per minute (BPM) input streams of the monitored subject.

According to some embodiments, a glucose event detection method may further comprise: (1) feeding to the artificial RNN architecture model, as input data, variable BPM input streams of the monitored subject, for the model to generate corresponding variable BGL model output streams predictions; (2) applying a loss function for measuring the “error” in the model's BGL output streams predictions, relative to time-aligned and labeled variable BGL output streams from a continuous blood glucose monitoring (CGM) device concurrently monitoring the subject; and (3) updating weight values of the RNN model based on the loss function measured “error”, to gradually reduce that error.

Functions, operations, components and/or features described herein with reference to one or more embodiments, may be combined or otherwise utilized with one or more other functions, operations, components and/or features described herein with reference to one or more other embodiments, or vice versa.

While certain features of the invention have been illustrated and described herein, many modifications, substitutions, changes, and equivalents will now occur to those skilled in the art. It is, therefore, to be understood that the appended claims are intended to cover all such modifications and changes as fall within the true spirit of the invention.

1. A glucose event detection system, comprising:
an artificial recurrent neural network (RNN) architecture of long short-term memory (LSTM) cells, configured to generate, for a monitored subject, variable blood glucose level (BGL) output streams predictions for respective, system fed, variable heart beats per minute (BPM) input streams of the monitored subject.

2. The system according to claim 1, further comprising a computer readable medium including instructions for a supervised training mechanism for, iteratively: (1) feeding to said artificial RNN architecture model, as input data, variable BPM input streams of the monitored subject, for the model to generate corresponding variable BGL model output streams predictions; (2) applying a loss function for measuring the “error” in the model's BGL output streams predictions, relative to time-aligned and labeled variable BGL output streams from a continuous blood glucose monitoring (CGM) device concurrently monitoring the subject; and (3) updating weight values of said RNN model based on said loss function measured “error”, to gradually reduce that error.

3. The system according to claim 2, further comprising a photoplethysmogram (PPG) based device for monitoring the heart rate of the subject and generating the timestamped variable BPM input streams for: (1) training of said artificial RNN model; and (2) feeding to the trained said artificial RNN model, to generate respective variable blood glucose level (BGL) output streams predictions based thereof.

4. The system according to claim 3, wherein said RNN model's blood glucose level (BGL) predictions output streams are postprocessed to detect a specific value or values-trends indicative of a glucose event; and, wherein detection of a glucose event triggers the relaying of a notification including one or more of said RNN model's blood glucose level (BGL) predictions associated with the detected event.

5. The system according to claim 4, further comprising an accelerometer concurrently monitoring the subject, wherein said accelerometer output values are utilized for mitigating false-positive alerts detected within said RNN model's BGL level outputs.

6. The system according to claim 5, wherein said accelerometer output values are analyzed to detect ‘higher than normal’ values occurring concurrently with glucose events detected within said RNN model's BGL values outputs; and, wherein at least part of said RNN model's detected glucose events—time overlapping with ‘higher than normal’ accelerometer values—are not regarded as glucose events, due to their overlapping time occurrence with higher physical activity indications that are based on said accelerometer output values.

7. The system according to claim 2, wherein said CGM device is a noninvasive wearable CGM device.

8. The system according to claim 2, wherein the monitored subject's BPM inputs to said RNN model are preprocessed to take the form of a scalar—representing the heart rate value of the subject at a specific timepoint.

9. The system according to claim 2, wherein the monitored subject's BPM inputs to said RNN model are preprocessed to take the form of a vector—representing several consecutive heart rate values of the subject.

10. The system according to claim 2, wherein a first network section of said RNN consists of a memory-based architecture, for connecting between BPM input series and BGL output series.

11. The system according to claim 10, wherein a second network section of said RNN consists of one or more fully-connected layers, for yielding a final output of the model—a predicted BGL value of the monitored subject at a specific timepoint—based on the output of said memory-based section.

12. The system according to claim 11, wherein the variable heart beats per minute (BPM) input streams of the monitored subject are pre-processed prior to being fed to said RNN by application of a logarithmic function on the raw input values.

13. The system according to claim 12, wherein said first network section of said RNN includes convolutional layers.

14. The system according to claim 11, wherein the variable heart beats per minute (BPM) input streams of the monitored subject are pre-processed prior to being fed to said RNN by application of a learnable embeddings function.

15. The system according to claim 14, wherein said first network section of said RNN includes non-convolutional layers.

16. The system according to claim 10, wherein said memory-based section of said RNN, outputs a single prediction over multiple input data timestamps.

17. The system according to claim 10, wherein said memory-based section of said RNN, outputs multiple predictions, corresponding to multiple input data timestamps.

18. The system according to claim 4, wherein said RNN model's BGL values outputs are analyzed for the detection of glucose events, based on a classification scheme, selected from the group including: (1) detection of an anomaly, based on one or more peaks in said RNN model's BGL values outputs—wherein a peak is defined as an increase beyond a threshold size of the BGL values outputs, the increase occurring within a time period shorter than a threshold length time period; (2) classifying the anomaly as either a food intake related or physical activity related anomaly; and (3) reacting only to anomalies classified as food intake related.

19. A glucose event detection method, comprising:
feeding variable heart beats per minute (BPM) input streams of a monitored subject to an artificial recurrent neural network (RNN) architecture of long short-term memory (LSTM) cells, configured to generate, time-respective variable blood glucose level (BGL) output streams predictions for the fed variable heart beats per minute (BPM) input streams of the monitored subject.

20. The method according to claim 19, further comprising:
(1) feeding to the artificial RNN architecture model, as input data, variable BPM input streams of the monitored subject, for the model to generate corresponding variable BGL model output streams predictions; (2) applying a loss function for measuring the “error” in the model's BGL output streams predictions, relative to time-aligned and labeled variable BGL output streams from a continuous blood glucose monitoring (CGM) device concurrently monitoring the subject; and (3) updating weight values of the RNN model based on the loss function measured “error”, to gradually reduce that error.
